(19) United States
(12) Patent Application Publication
(10) Pub. No.: US 2023/0015547 A1
(43) Pub. Date: Jan. 19, 2023

(54) FILTRATION ENCLOSURE FOR VENTILATION BAGS

(71) Applicant: JAMIE TART, COLUMBIA, SC (US)

(72) Inventor: JAMIE TART, COLUMBIA, SC (US)

(21) Appl. No.: 17/373,841

(22) Filed: Jul. 13, 2021

(51) Int. Cl. A62B 18/02; A62B 7/10; A62B 9/04; A62B 18/08; A62B 9/02

(52) U.S. Cl. CPC A62B 18/02; A62B 7/10; A62B 9/04; A62B 18/08; A62B 9/02

(57) ABSTRACT

The filtration enclosure for ventilation bags is a manually operated respirator. The filtration enclosure for ventilation bags is adapted for use with a patient. The filtration enclosure for ventilation bags is a breathing assistance device that maintains a positive pressure within the lungs of the patient. The filtration enclosure for ventilation bags incorporates a bag valve mask respirator and a containment structure. The containment structure forms a protection space that encloses the bag valve mask respirator such that the bag valve mask respirator can operate while within the containment space. The containment structure is formed as a gas permeable structure. The containment structure forms a filter that filters microorganisms from the gas that flows through the boundary surface formed by the containment structure.

### CROSS REFERENCES TO RELATED APPLICATIONS

Not Applicable

### STATEMENT REGARDING FEDERALLY SPONSORED RESEARCH

Not Applicable

### REFERENCE TO APPENDIX

Not Applicable

### BACKGROUND OF THE INVENTION

### Field of the Invention

The present invention relates to the field of life saving equipment including respiratory apparatus. (A62B7/00)

### SUMMARY OF INVENTION

The filtration enclosure for ventilation bags is a manually operated respirator. The filtration enclosure for ventilation bags is adapted for use with a patient. The filtration enclosure for ventilation bags is a breathing assistance device that maintains a positive pressure within the lungs of the patient. The filtration enclosure for ventilation bags comprises a bag valve mask respirator and a containment structure. The containment structure forms a protection space that encloses the bag valve mask respirator such that the bag valve mask respirator can operate while within the containment space. The containment structure is formed as a gas permeable structure. The containment structure forms a filter that filters microorganisms from the gas that flows through the boundary surface formed by the containment structure.

These together with additional objects, features and advantages of the filtration enclosure for ventilation bags will be readily apparent to those of ordinary skill in the art upon reading the following detailed description of the presently preferred, but nonetheless illustrative, embodiments when taken in conjunction with the accompanying drawings.

In this respect, before explaining the current embodiments of the filtration enclosure for ventilation bags in detail, it is to be understood that the filtration enclosure for ventilation bags is not limited in its applications to the details of construction and arrangements of the components set forth in the following description or illustration. Those skilled in the art will appreciate that the concept of this disclosure may be readily utilized as a basis for the design of other structures, methods, and systems for carrying out the several purposes of the filtration enclosure for ventilation bags.

It is therefore important that the claims be regarded as including such equivalent construction insofar as they do not depart from the spirit and scope of the filtration enclosure for ventilation bags. It is also to be understood that the phraseology and terminology employed herein are for purposes of description and should not be regarded as limiting.

### BRIEF DESCRIPTION OF DRAWINGS

The accompanying drawings, which are included to provide a further understanding of the invention are incorporated in and constitute a part of this specification, illustrate an embodiment of the invention and together with the description serve to explain the principles of the invention. They are meant to be exemplary illustrations provided to enable persons skilled in the art to practice the disclosure and are not intended to limit the scope of the appended claims.

FIG. 1 is a front view of an embodiment of the disclosure.

FIG. 2 is a detail view of an embodiment of the disclosure.

FIG. 3 is an in-use view of an embodiment of the disclosure.

### DETAILED DESCRIPTION OF THE EMBODIMENT

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description.

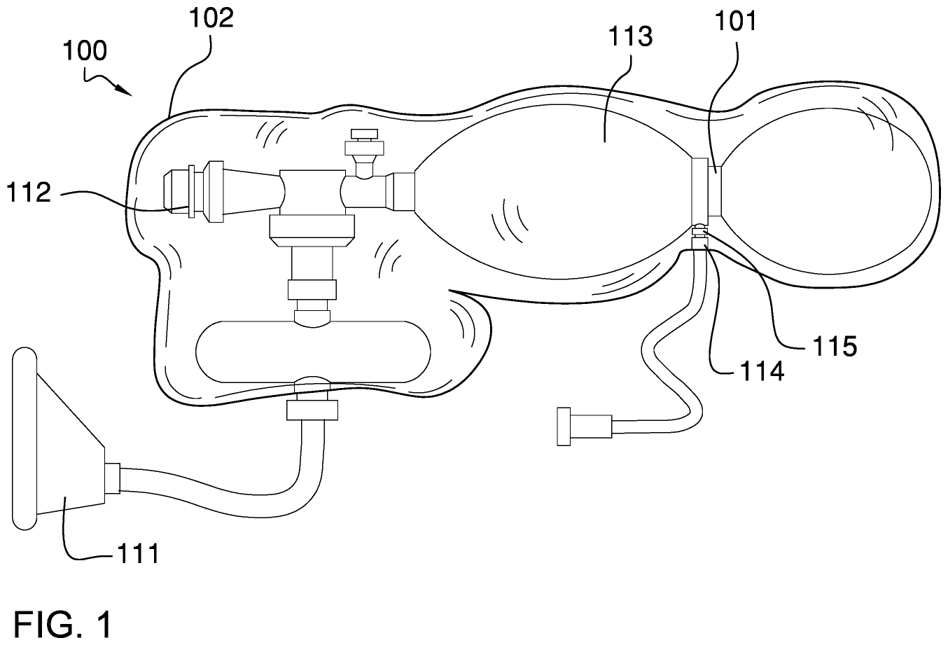

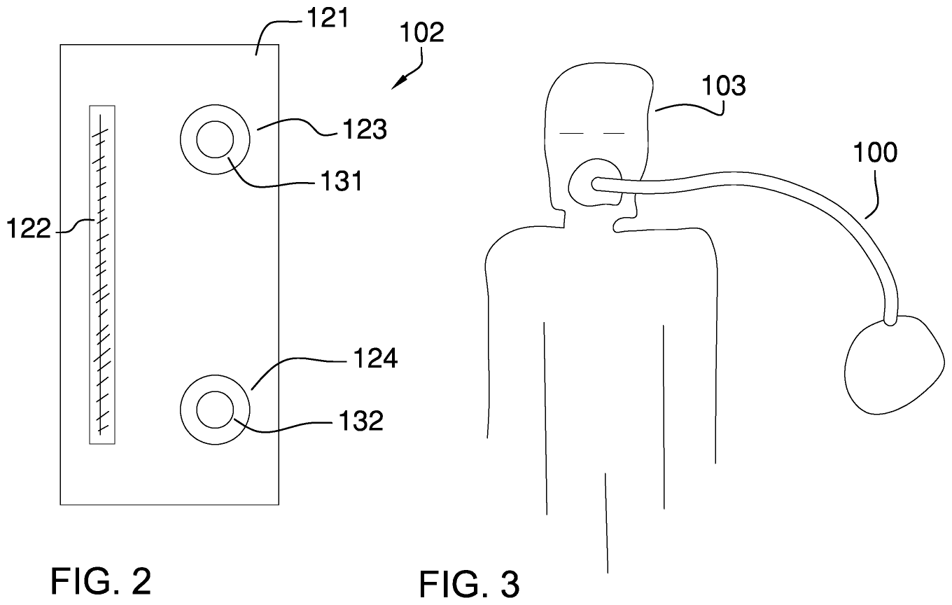

Detailed reference will now be made to one or more potential embodiments of the disclosure, which are illustrated in FIGS. 1 through 3.

The filtration enclosure for ventilation bags 100 (hereinafter invention) is a manually operated respirator. The invention 100 is adapted for use with a patient 103. The invention 100 is a breathing assistance device that maintains a positive pressure within the lungs of the patient 103. The invention 100 comprises a bag valve mask respirator 101 and a containment structure 102. The bag valve mask respirator 101 provides breathing assistance by providing gas under pressure to the patient 103. The containment structure 102 forms a protection space that encloses the bag valve mask respirator 101 such that the bag valve mask respirator 101 can operate while within the containment space. The containment structure 102 is formed as a gas permeable structure. The containment structure 102 forms a filter that filters microorganisms from the gas that flows through the boundary surface formed by the containment structure 102.

The patient 103 is an individual who requires the services of the bag valve mask respirator 101. The patient 103 is defined elsewhere in this disclosure.

The bag valve mask respirator 101 is a medical device. The bag valve mask respirator 101 is a mechanical device. The bag valve mask respirator 101 is a handheld device. The bag valve mask respirator 101 is a manually operated device. The bag valve mask respirator 101 is adapted for use with a patient 103. The bag valve mask respirator 101 generates a breathable gas under pressure that is provided to the patient 103. The bag valve mask respirator 101 regulates the pressure of the breathable gas to the patient 103. The bag valve mask respirator 101 replaces the breathable gas provided to the patient 103 with fresh breathable gas derived from an externally provided source. The bag valve mask respirator 101 is defined elsewhere in this disclosure. The bag valve mask respirator 101 comprises a mask structure 111, a peep valve structure 112, a ventilation bag structure 113, and a gas intake structure 114.

The mask structure 111 is a mechanical structure. The mask structure 111 forms the mask of the bag valve mask respirator 101. The mask structure 111 is a respiratory mask that delivers the breathable gas to the patient 103. The mask structure 111 removably attaches to the patient 103. The mask structure 111 forms a fluidic connection with the ventilation bag structure 113. The mask structure 111 receives the pressurized breathable gas from the ventilation bag structure 113.

The peep valve structure 112 is a mechanical structure. The peep valve structure 112 forms the peep valve of the bag valve mask respirator 101. The peep valve structure 112 is a pressure regulator that maintains breathable gas generated under pressure by the ventilation bag structure 113 at a constant gas pressure that is appropriate for the patient 103. The peep valve structure 112 inserts into the gas flow path between the mask structure 111 and the ventilation bag structure 113.

The ventilation bag structure 113 is a mechanical structure. The ventilation bag structure 113 forms the mask of the bag valve mask respirator 101. The ventilation bag structure 113 stores the breathable gas in anticipation of use. The ventilation bag structure 113 is an elastic structure. The ventilation bag structure 113 is compressible such that the storage volume of the ventilation bag structure 113 is reducible. The ventilation bag structure 113 is in its relaxed shape when the ventilation bag structure 113 is at its full containment volume. The compression of the ventilation bag structure 113 places the breathable gas contained within the ventilation bag structure 113 under pressure such that the breathable gas is transported to the mask structure 111. After the compression of the ventilation bag structure 113 is complete, the ventilation bag structure 113 returns to its relaxed shape drawing fresh breathable gas into the ventilation bag structure 113 through the gas intake structure 114.

The gas intake structure 114 is a gas port. The gas intake structure 114 forms a fluidic connection between a gas source and the interior volume of the ventilation bag structure 113. The gas intake structure 114 further comprises a check valve 115. The check valve 115 is a valve that inserts into the gas intake structure 114. The check valve 115 prevents the backflow of gas from the ventilation bag structure 113 back to the gas source.

The containment structure 102 forms a protected space that encloses the bag valve mask respirator 101. The containment structure 102 is a textile based structure. The containment structure 102 is a flexible structure. The containment structure 102 is a gas permeable structure. The containment structure 102 forms a filter the removes microorganisms from any gas that flows through the boundary of the containment structure 102. The containment structure 102 contains the bag valve mask respirator 101 such that the bag valve mask respirator 101 is fully operable while contained within the containment structure 102. The containment structure 102 comprises a shell structure 121, a fastening structure 122, a gas intake aperture 123, and a gas discharge aperture 124.

The shell structure 121 is a textile based structure. The shell structure 121 is a flexible structure. The shell structure 121 forms a bag structure that encloses the bag valve mask respirator 101. The shell structure 121 is a gas permeable structure. The shell structure 121 forms the filter that removes microorganisms from the gas that flow through the textile structure of the shell structure 121. The shell structure 121 is formed with an aperture such that the bag valve mask respirator 101 can be inserted into and withdrawn from the shell structure 121. In the first potential embodiment of the disclosure, the shell structure 121 forms an N95 filter.

The fastening structure 122 is a fastening device. The fastening structure 122 control access into the shell structure 121 through the formed aperture. In the first potential embodiment of the disclosure, the fastening structure 122 is a zipper.

The gas intake aperture 123 is an aperture that allows the gas intake structure 114 to insert through the shell structure 121. The gas intake aperture 123 further comprises a gas intake grommet 131. The gas intake grommet 131 is a mechanical device that protects the perimeter of the gas intake aperture 123. The gas intake grommet 131 forms the space through which the gas intake structure 114 inserts. The gas intake grommet 131 forms a fluid impermeable seal between the gas intake structure 114 and the gas intake aperture 123.

The gas discharge aperture 124 is an aperture that allows the fluidic connection between the ventilation bag structure 113 and the mask structure 111 to insert through the shell structure 121. The gas discharge aperture 124 further comprises a gas discharge grommet 132. The gas discharge grommet 132 is a mechanical device that protects the perimeter of the gas discharge aperture 124. The gas discharge grommet 132 forms the space through which the fluidic connection inserts. The gas discharge grommet 132 forms a fluid impermeable seal between the fluidic connection and the gas discharge aperture 124.

The following definitions were used in this disclosure:

Atmosphere: As used in this disclosure, the atmosphere refers to a blanket of gases (primarily nitrogen and oxygen) that surround the earth. Typical atmospheric conditions are approximated and characterized as the normal temperature and pressure. Atmospheric gases are commonly called air.

Bag: As used in this disclosure, a bag is a container made of a flexible material. The bag has a single opening which allows the bag to receive the items to be contained.

Bag Mask Valve Respirator: As used in this disclosure, the bag mask valve respirator is a manually operated respiration device. The bag mask valve respirator is adapted for use with a patient. The bag mask valve respirator generates a positive pressure within the lungs of the patient to assist the breathing of the patient. The bag mask valve respirator comprises a mask, a bag, a peep valve, and a gas intake. The mask covers the mouth and nose of the patient and forms a fluidic connection that guides the pressurized gas into the patient. The bag is a hollow elastic structure. In its relaxed shape, the bag is expanded to its maximum volume. The bag forms a fluidic connection with the mask. When the bag is compressed, the pressure of the gas contained in the bag is increased such that the gas is forced through the mask into the patient such that a positive gas pressure is maintained in the lungs of the patient. The PEEP valve is a pressure regulator that inserts into the gas flow between the mask and the bag such that a constant pressure is maintained within the lungs of the patient. The gas intake is a port that forms a fluidic connection between the gas source (such as the atmosphere or an oxygen source) and the bag. The gas intake allows gas to flow from the gas source into the bag as the bag returns to its relaxed shape. The gas intake incorporates a check valve such that the gas will only flow from the gas source into the bag.

Barrier: As used in this disclosure, a barrier is a physical obstacle that forms a boundary between a first space and a second space. The barrier prevents the passage of an object between the first space and the second space.

Check Valve: As used in this disclosure, a check valve is a valve that permits the flow of fluid in a single direction. Within selected potential embodiments of this disclosure, the check valve is a commercially available product that is selected from the group consisting of a ball valve and a Tesla valve.

Elastic: As used in this disclosure, an elastic is a material or object that deforms when a force is applied to it and that is able to return to its relaxed shape after the force is removed. A material that exhibits these qualities is also referred to as an elastomeric material. A material that does not exhibit these qualities is referred to as inelastic or an inelastic material.

Elastic Nature: As used in this disclosure, an elastic nature refers to a flexible structure that returns to its relaxed shape after the flexible structure has been deformed.

Fastener: As used in this disclosure, a fastener is a device that is used to join or affix two objects. Fasteners generally comprise a first element which is attached to the first object and a second element which is attached to the second object such that the first element and the second element join to removably attach the first object and the second object. Common fasteners include, but are not limited to, hooks, zippers, magnets, snaps, buttons, buckles, quick release buckles, or hook and loop fasteners. A fastener is often referred to as a fastening device.

Filter: As used in this disclosure, a filter is a mechanical device that is used to separate solids that are suspended in a liquid or a gas.

Flexible: As used in this disclosure, flexible refers to an object or material that will deform when a force is applied to it but that will not necessarily return to its original shape when the deforming force is removed.

Flow: As used in this disclosure, a flow refers to the passage of a fluid past a fixed point. This definition considers bulk solid materials as capable of flow.

Fluid: As used in this disclosure, a fluid refers to a state of matter wherein the matter is capable of flow and takes the shape of a container it is placed within. The term fluid commonly refers to a liquid or a gas.

Fluidic Connection: As used in this disclosure, a fluidic connection refers to a tubular structure that transports a fluid from a first object to a second object. Methods to design and use a fluidic connections are well-known and documented in the mechanical, chemical, and plumbing arts.

Force: As used in this disclosure, a force refers to a net (or unopposed) measurable interaction that changes the direction of motion of an object, the velocity of motion of an object, the momentum of an object, or the stress within an object. The term work refers to a measure of the amount of energy that is transferred through the application of a force over a distance. The term power refers to a measure of the amount of energy that is transferred over a period of time.

Gas: As used in this disclosure, a gas refers to a state (phase) of matter that is fluid and that fills the volume of the structure that contains it. Stated differently, the volume of a gas always equals the volume of its container.

Grommet: As used in this disclosure, a grommet is an eyelet placed in a hole in a textile, sheet, or panel that protects a rope hook or cable passed through it and to protect the textile, sheet, or panel from being torn. See bushing and flange.

Inelastic Nature: As used in this disclosure, an inelastic nature refers to a flexible structure that maintains its new shape after the flexible structure has been deformed.

Liquid: As used in this disclosure, a liquid refers to a state (phase) of matter that is fluid and that maintains, for a given pressure, a fixed volume that is independent of the volume of the container.

Mask: As used in this disclosure, a mask is a covering for the face of a person. A mask filters air as it passes through the nose and mouth of a patient. A mask that routes and concentrates a flow of air to the nose and mouth of a patient is called a respiratory mask.

Microorganism: As used in this disclosure, a microorganism is an organism too small to be viewed by the unaided eye. Microorganisms are typically single celled organisms such as bacteria, yeast, viruses, protozoa, fungi and algae. A pathogen refers to a microorganism that has the potential to cause illness or disease.

N95 Filter: As used in this disclosure, an N95 filter is a surface filter designed to remove particulates from an air flow. The established performance standard for the N95 filter requires that the N95 filter be capable of removing 95% of the particulates having a diameter of greater than or equal to 300 nanometers from the air flow. As a practical matter, most N95 filters remove over 99% (a published estimate that was current as this definition is written has 99.8%) of the particulates having a diameter of greater than or equal to 100 nanometers from the air flow. An N95 respirator, or less formally an N95 mask, is a respirator that filters the flow of breathing air through an N95 filter. An N99 filter is rated as removing over 99% of the particulates having a diameter of greater than or equal to 300 nanometers from the air flow.

Patient: As used in this disclosure, a patient is a person who is designated to receive a medical treatment, therapy or service. The term patient may be extended to an animal when used within the context of the animal receiving veterinary treatment or services.

PEEP: As used in this disclosure, PEEP stands for Positive End Expiratory Pressure. PEEP is the pressure in the lungs above atmospheric pressure. Specifically, this definition refers to extrinsic PEEP which is a mechanically applied pressure, usually provided through a ventilator that prevents or mitigates alveolar collapse. PEEP is closely related to CPAP.

PEEP Valve: As used in this disclosure, a PEEP valve is an acronym for a positive end expiratory pressure valve. The positive end expiratory pressure valve is a valve that is used to maintain used to maintain a positive air pressure within the respiratory system of a patient that is greater than atmospheric pressure. Among the several purposes of a PEEP valve, PEEP valves are used with ventilators to prevent the lungs from collapsing during expiration. A PEEP valve will often use a mechanical impedance system to regulate the pressure within the respiratory system. PEEP valves with an adjustable pressure mechanism are also available. Adjustable PEEP valves can be identified with a threaded cap on the end of the valve that is used to adjust the tension on a spring.

Phase: As used in this disclosure, phase refers to the state of the form of matter. The common states of matter are solid, liquid, gas, and plasma.

Pressure: As used in this disclosure, pressure refers to a measure of force per unit area.

Protected Space: As used in this disclosure, a protected space is a negative space within which an object is stored. The protected space is enclosed by a barrier structure that: a) prevents damage to the object contained within the protected space; b) maintains an environment suitable within the protected space that is appropriate for the object; or, c) protects the object within the protected space from potential dangers that are outside of the protected space.

Relaxed Shape: As used in this disclosure, a structure is considered to be in its relaxed state when no shear, strain, or torsional forces are being applied to the structure.

Seal: As used in this disclosure, a seal is a structure that forms a fluid impermeable barrier between two objects.

Sheeting: As used in this disclosure, a sheeting is a material, such as a paper, textile, a plastic, or a metal foil, in the form of a thin flexible layer or layers. The sheeting forms a disk structure. The two surfaces of the sheeting with the greatest surface area are called the faces of the sheeting.

Solid: As used in this disclosure, a solid refers to a state (phase) of matter that: 1) has a fixed volume; and, 2) does not flow.

Spacer: As used in this disclosure, a spacer is a prism-shaped disk that is formed with a cylindrical negative space that allows a shaft to be inserted through the faces of the disk structure of the spacer. A spacer is further defined with an inner diameter. A spacer is often referred to as a washer. A spacer formed from an elastic material is called an o-ring.

Such As: As used in this disclosure, the term “such as” is a conjunction that relates a first phrase to a subsequent phrase. The term “such as” is used to introduce representative examples of structures that meet the requirements of the first phrase. As a first example of the use of the term “such as,” the phrase: “the first textile attaches to the second textile using a fastener such as a hook and loop fastener” is taken to mean that a hook and loop fastener is suitable to use as the fastener but is not meant to exclude the use of a zipper or a sewn seam. As a second example of the use of the term “such as,” the phrase: “the chemical substance is a halogen such as chlorine or bromine” is taken to mean that either chlorine or bromine are suitable for use as the halogen but is not meant to exclude the use of fluorine or iodine.

Such That: As used in this disclosure, the term “such that” is a conjunction that relates a first phrase to a subsequent phrase. The term “such that” is used to place a further limitation or requirement to the first phrase. As a first example of the use of the term “such that,” the phrase: “the door attaches to the wall such that the door rotates relative to the wall” requires that the attachment of the door allows for this rotation. As a second example of the use of the term “such that,” the phrase: “the chemical substance is selected such that the chemical substance is soluble in water” requires that the selected chemical substance is soluble in water. As a third example of the use of the term “such that,” the phrase: “the lamp circuit is constructed such that the lamp circuit illuminates when the lamp circuit detects darkness” requires that the lamp circuit: a) detect the darkness; and, b) generate the illumination when the darkness is detected.

Surface Filter: As used in this disclosure, a surface filter is a type of filter wherein the fluid is passed through a surface or membrane, such as a screen or paper that allows for the passage of the fluid but blocks the passage of larger particles that may be suspended in the fluid. The construction of a surface filter would allow for the passage of the fluid through several filter surfaces in one filtration unit.

Textile: As used in this disclosure, a textile is a material that is woven, knitted, braided or felted. Synonyms in common usage for this definition include fabric and cloth. The two surfaces of the textile with the greatest surface area are called the faces of the textile.

Zipper: As used in this disclosure, a zipper is a fastening device comprising a first chain tape, a second chain tape, and a zipper pull. The first chain tape and the second chain tape are textile webbings formed with interlocking components that form a chain. The chain is opened and closed by pulling a slide, called a zipper pull, over the first chain tape, a second chain tape. The individual elements of the chain are called the teeth of the chain.

With respect to the above description, it is to be realized that the optimum dimensional relationship for the various components of the invention described above and in FIGS. 1 through 3 include variations in size, materials, shape, form, function, and manner of operation, assembly and use, are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

What is claimed is:

1. A filtration enclosure for ventilation bags comprising
a bag valve mask respirator and a containment structure;
wherein the containment structure forms a protection space that encloses the bag valve mask respirator.

2. The filtration enclosure for ventilation bags according to claim 1
wherein the filtration enclosure for ventilation bags is a manually operated respirator;
wherein the filtration enclosure for ventilation bags is adapted for use with a patient;
wherein the filtration enclosure for ventilation bags is a breathing assistance device that maintains a positive pressure of a breathable gas.

3. The filtration enclosure for ventilation bags according to claim 2
wherein the containment structure is formed as a gas permeable structure;
wherein the containment structure forms a filter that filters microorganisms from the gas that flows through the boundary surface formed by the containment structure.

4. The filtration enclosure for ventilation bags according to claim 3
wherein the bag valve mask respirator is a medical device;
wherein the bag valve mask respirator is a mechanical device;
wherein the bag valve mask respirator is a handheld device;
wherein the bag valve mask respirator is a manually operated device;
wherein the bag valve mask respirator generates the breathable gas under pressure that is provided to the patient;
wherein the bag valve mask respirator regulates the pressure of the breathable gas to the patient;
wherein the bag valve mask respirator replaces the breathable gas provided to the patient with fresh breathable gas derived from an externally provided source.

5. The filtration enclosure for ventilation bags according to claim 4
wherein the containment structure forms a protection space that encloses the bag valve mask respirator such that the bag valve mask respirator can operate while within the containment space.

6. The filtration enclosure for ventilation bags according to claim 5
wherein the containment structure forms a protected space that encloses the bag valve mask respirator;
wherein the containment structure is a textile based structure;
wherein the containment structure is a flexible structure;
wherein the containment structure is a gas permeable structure;
wherein the containment structure forms a filter the removes microorganisms from any gas that flows through the boundary of the containment structure;
wherein the containment structure contains the bag valve mask respirator such that the bag valve mask respirator is fully operable while contained within the containment structure.

7. The filtration enclosure for ventilation bags according to claim 6 wherein the containment structure is an N95 filter.

8. The filtration enclosure for ventilation bags according to claim 7
wherein the bag valve mask respirator comprises a mask structure, a peep valve structure, a ventilation bag structure, and a gas intake structure;
wherein the mask structure is a mechanical structure;
wherein the mask structure forms a fluidic connection with the ventilation bag structure;
wherein the peep valve structure inserts into the gas flow path between the mask structure and the ventilation bag structure.

9. The filtration enclosure for ventilation bags according to claim 8
wherein the containment structure comprises a shell structure, a fastening structure, a gas intake aperture, and a gas discharge aperture;
wherein the fastening structure, the gas intake aperture, and the gas discharge aperture are formed in the shell structure.

10. The filtration enclosure for ventilation bags according to claim 9
wherein the mask structure forms the mask of the bag valve mask respirator;
wherein the mask structure is a respiratory mask that delivers the breathable gas to the patient;
wherein the mask structure removably attaches to the patient;
wherein the mask structure receives the pressurized breathable gas from the ventilation bag structure.

11. The filtration enclosure for ventilation bags according to claim 10
wherein the peep valve structure is a mechanical structure;
wherein the peep valve structure forms the peep valve of the bag valve mask respirator;
wherein the peep valve structure is a pressure regulator that maintains breathable gas generated under pressure by the ventilation bag structure at a constant gas pressure that is appropriate for the patient.

12. The filtration enclosure for ventilation bags according to claim 11
wherein the ventilation bag structure is a mechanical structure;
wherein the ventilation bag structure forms the mask of the bag valve mask respirator;
wherein the ventilation bag structure stores the breathable gas in anticipation of use;
wherein the ventilation bag structure is an elastic structure;
wherein the ventilation bag structure is compressible such that the storage volume of the ventilation bag structure is reducible;
wherein the ventilation bag structure is in its relaxed shape when the ventilation bag structure is at its full containment volume.

13. The filtration enclosure for ventilation bags according to claim 12
wherein the gas intake structure is a gas port;
wherein the gas intake structure forms a fluidic connection between a gas source and the interior volume of the ventilation bag structure.

14. The filtration enclosure for ventilation bags according to claim 13
wherein the gas intake structure further comprises a check valve;
wherein the check valve is a valve that inserts into the gas intake structure;
wherein the check valve prevents the backflow of gas from the ventilation bag structure back to the gas source.

15. The filtration enclosure for ventilation bags according to claim 14
wherein the shell structure is a textile based structure;
wherein the shell structure is a flexible structure;
wherein the shell structure forms a bag structure that encloses the bag valve mask respirator;
wherein the shell structure is a gas permeable structure;
wherein the shell structure forms the filter that removes microorganisms from the gas that flow through the textile structure of the shell structure;
wherein the shell structure is formed with an aperture such that the bag valve mask respirator can be inserted into and withdrawn from the shell structure;
wherein the fastening structure is a fastening device.

16. The filtration enclosure for ventilation bags according to claim 15
wherein the fastening structure control access into the shell structure through the formed aperture;
wherein in the first potential embodiment of the disclosure, the fastening structure is a zipper;
wherein the gas intake aperture is an aperture that allows the gas intake structure to insert through the shell structure;
wherein the gas intake aperture further comprises a gas intake grommet;
wherein the gas intake grommet is a mechanical device that protects the perimeter of the gas intake aperture;
wherein the gas intake grommet forms the space through which the gas intake structure inserts;
wherein the gas intake grommet forms a fluid impermeable seal between the gas intake structure and the gas intake aperture.

17. The filtration enclosure for ventilation bags according to claim 16
wherein the gas discharge aperture is an aperture that allows the fluidic connection between the ventilation bag structure and the mask structure to insert through the shell structure;
wherein the gas discharge aperture further comprises a gas discharge grommet;
wherein the gas discharge grommet is a mechanical device that protects the perimeter of the gas discharge aperture;
wherein the gas discharge grommet forms the space through which the fluidic connection inserts;
wherein the gas discharge grommet forms a fluid impermeable seal between the fluidic connection and the gas discharge aperture.
